(19) United States
(12) Patent Application Publication
(10) Pub. No.: US 2011/0257690 A1
(43) Pub. Date: Oct. 20, 2011

(54) Transverse and Sagittal Adjusting Screw

(75) Inventor: William A. Rezach, Atoka, TN (US)

(73) Assignee: WARSAW ORTHOPEDIC, INC., Warsaw, IN (US)

(21) Appl. No.: 12/763,564

(22) Filed: Apr. 20, 2010

(51) Int. Cl. A61B 17/86

(52) U.S. Cl. 606/302; 606/308

(57) ABSTRACT

A bone anchor assembly is provided, which may be used in cervical, thoracic, lumbar or sacral areas of the spine or other orthopedic locations. The anchor assembly includes a bone anchor, a receiver mounted to the bone anchor, a saddle within the receiver, a spacer within the receiver, and an engaging member. The receiver extends along a central longitudinal axis proximally away from the bone anchor. A rod or other elongated connecting element is received in a passage of the receiver in contact with the saddle, and the engaging member engages the connecting element against the saddle, which engages the saddle against the spacer, which in turn engages the proximal head of the bone anchor in the receiver. The orientation of the saddle in the receiver is adjustable to correspond to the orientation of the connecting element relative to the central longitudinal axis of the receiver.

### BACKGROUND

The present invention concerns bone anchors and anchor assemblies, particularly useful for engagement to vertebrae. In a particular embodiment, the invention contemplates a bone anchor assembly with an adjustable saddle to secure an elongate connecting element, such as a spinal rod, along the spinal column.

Several techniques and systems have been developed for correcting and stabilizing the spine and for facilitating fusion at various levels of the spine. In one type of system, an elongated rod is disposed longitudinally along the length of the spine or several vertebrae of the spinal column. The rod may be bent to correspond to the normal or desired curvature of the spine in the particular region being instrumented. For example, the rod can be bent or angled to form a normal kyphotic curvature for the thoracic region of the spine, or a lordotic curvature for the lumbar region. In accordance with such a system, the rod is engaged to various vertebrae along the length of the spinal column by way of a number of fixation elements. A variety of fixation elements can be provided which are configured to engage specific portions of the vertebra. For instance, one such fixation element is a hook that is configured to engage the lamina of the vertebra. Another type of fixation element is a spinal screw which can be threaded into various aspects of the vertebral bone, such as the pedicle.

In one typical procedure utilizing a bendable, angled or linear rod, one or more of the rods is situated on one or both of the opposite sides of the spine or spinous processes. A plurality of bone screws are threadingly engaged to several vertebral bodies, such as to the pedicles of these vertebrae. One or more of the bone screws are maneuvered to manipulate the position or orientation of the vertebral body or bodies to which the bone screw is engaged. The rod(s) are connected or affixed to the plurality of bone screws to apply and maintain corrective and stabilizing forces to the spine.

The bone anchors in spinal procedures can have receivers with channels for the elongated rod or other member that, in some bone anchors, open upward, i.e. directly away from the bone to which the anchor is attached. Other bone anchors utilize channels that open along the medial or lateral side of the anchor to receive the rod. It is desirable in some procedures to utilize a bone anchor where the bone engaging portion of the bone anchor and the receiver are fixed relative to one another so that the forces applied to the receiver are effectively transferred to the vertebra. However, the relative positions of the vertebra and the receiver of the bone anchor may require contouring, bending, and/or angling of the rod through the channel of the bone anchor, which can result in a less than optimal fit between the anchor and the rod, creating undesirable stress concentrations in the rod, bone anchor and/or bony structure. Additional improvements in the bone anchor and rod interface in spinal systems are still needed.

### SUMMARY

According to one aspect a bone anchor assembly is disclosed that includes a receiver extending along a central longitudinal axis between a proximal end and an opposite distal end. The receiver includes a distal portion defining a receptacle opening at the distal end and a pair of arms extending from the distal portion along the central longitudinal axis. A bone anchor is included that has a distal bone engaging portion and a head at a proximal end of the distal bone engaging portion. A cap including an upper surface extending to a lower surface. The lower surface of the cap includes an interior cavity sized and configured to receive the head of the bone anchor such that the bone anchor can pivot along a first axis. The cap is positioned in the receptacle of the receiver. A spacer is positioned in an aperture in the cap such that an upper portion of the spacer protrudes outwardly from the cap and a lower portion of the spacer is connected with an upper surface of the head of the bone anchor.

A saddle is positioned in the passage of the receiver adjacent to a bottom surface of the receiver. The saddle includes a proximal support surface and a distal surface opposite the proximal support surface. The upper portion of the spacer is in contact with the distal surface of the saddle. The spacer includes at least one edge operable to bite into the distal surface of the saddle to secure the saddle in a respective location within the receiver. In one form, the saddle is movable in the receiver so that the support surface parallels a longitudinal axis of a connecting element in orientations of the longitudinal axis of the connecting element that vary up to 30 degrees from an orthogonal orientation of said longitudinal axis of the connecting element with the central longitudinal axis of the receiver.

The cap includes a first set of apertures that are sized and configured to receive a first pin. The head of the bone anchor includes a passage aligned with the first set of apertures and the first pin is inserted through the first set of apertures and the passage thereby securing the cap to the bone anchor. The bone anchor pivots along the first axis by pivoting about the first pin. The cap also includes a second set of apertures and the distal portion of the receiver includes a third set of apertures. A second pin is inserted through a respective one of the second set of apertures and a respective one of the third set of apertures and a third pin is inserted through a respective one of the second set of apertures and the third set of apertures thereby securing the cap to the receiver.

Another aspect of the present invention discloses a bone anchor assembly that includes a receiver extending along a central longitudinal axis between a proximal end and an opposite distal end. The receiver includes a distal portion defining a receptacle opening at the distal end and a pair of arms extending from the distal portion along the central longitudinal axis on opposite sides of a passage. The receiver also includes a bottom surface extending along the passage between the pair of arms. The passage opens at opposite sides of the receiver between the pair of arms and the receptacle opens into the passage through the bottom surface.

The bone anchor assembly also includes a cap having a cavity located in a central portion of the cap. A bone anchor is included that has a distal bone engaging portion and a head at a proximal end of the distal bone engaging portion. The head is configured to be received in the cap through the cavity. A saddle is positioned in the passage of the receiver adjacent to the bottom surface of the receiver. The saddle includes a proximal support surface and a distal surface opposite the proximal support surface. A spacer is positioned between the saddle and the head of the bone anchor. A connecting element extends along a central longitudinal axis, wherein the connecting element is located in the passage and extends through the opposite sides of the receiver. An engaging member is engaged to the pair of arms to secure the connecting element against the proximal support surface of the saddle. The saddle engages the receiver and is limited to movement in the receiver in a single plane defined by the central longitudinal axis of the receiver and the central longitudinal axis of the connecting element while the bone engaging portion remains in the first orientation.

The pair of arms includes inner surfaces facing one another on opposite sides of the passage. The inner surfaces each include a groove formed therein that is curved between opposite ends of a respective one of the pair of arms so that the curve includes a most distal portion at the central longitudinal axis and the groove is curved proximally from the most distal portion toward the opposite ends of the respective arm. The saddle includes at least one ear on each side of the proximal support surface that are positioned in a respective one of the grooves. The ears are slidably movable along the respective one of the grooves.

Another aspect discloses a bone anchor assembly that includes a receiver extending along a central longitudinal axis between a proximal end and an opposite distal end. The receiver includes a distal portion defining a receptacle opening at the distal end and a pair of arms extending from the distal portion along the central longitudinal axis on opposite sides of a passage with the passage opening at opposite sides of the receiver, wherein the receiver includes a bottom surface extending along the passage between the pair of arms and the receptacle opens into the passage. A cap is included that has an upper surface extending down toward a lower surface, wherein the cap has an interior cavity in the lower surface of the cap. A bone anchor is included that has a distal bone engaging portion and a head at a proximal end of the distal bone engaging portion. The head is positioned in the cavity of the cap such that the bone anchor can pivot along a transverse plane.

A saddle is positioned in the passage of the receiver adjacent to the bottom surface of the receiver. The saddle includes a proximal support surface and a distal surface opposite the proximal support surface. A spacer is positioned in the receptacle of the receiver such that an upper surface of the spacer is in contact with a lower surface of the saddle and a lower surface of the spacer is in contact with an upper surface of the head. A connecting element extends along a central longitudinal axis and is located in the passage and extends through the opposite sides of the receiver. An engaging member is engaged to the pair of arms to secure the connecting element against the proximal support surface of the saddle. Force applied to the saddle is transferred to the spacer which in turn transfers force to the head of said bone anchor thereby preventing the bone anchor from further pivoting in the cap.

Related features, aspects, embodiments, objects and advantages of the present invention will be apparent from the following description.

### BRIEF DESCRIPTION OF THE DRAWINGS

FIG. 1 is a posterior elevation view of a spinal column segment with a spinal implant system engaged thereto.

FIG. 2 is a partial sectional view of one embodiment of a bone anchor assembly with the connecting element in a first orientation relative to the bone anchor.

FIG. 3 is a partial sectional view of the bone anchor assembly of FIG. 2 with the connecting element in a second orientation relative to the bone anchor.

FIG. 4 is a perspective view of another representative bone anchor assembly.

FIG. 5 is a perspective view of the bone anchor assembly illustrated in FIG. 4 with certain components broken away.

FIG. 6 is a perspective view of a bone anchor.

FIG. 7 is a perspective view of a screw cap.

FIG. 8 is a perspective view of a bone anchor, screw cap and spacer.

FIG. 9a is a partial perspective view of a bone anchor, screw cap, spacer and saddle.

FIG. 9b is a partial perspective view of a bone anchor, spacer, and saddle.

FIG. 10a is a perspective view of a receiver.

FIG. 10b is a top perspective view of the receiver.

FIG. 10c is a front view of the receiver.

FIG. 10d is a side view of the receiver.

FIG. 11a is a perspective view of a saddle comprising a portion of the bone anchor assembly.

FIG. 11b is a top plan view of the saddle of FIG. 11a.

FIG. 11c is a side elevation view of the saddle of FIG. 11a.

FIG. 11d is an end elevation view of the saddle of FIG. 11a.

FIG. 11e is a section view along line 11e-11e of FIG. 11b.

FIG. 11f is a section view along line 11f-11f of FIG. 11c.

FIG. 12 is a cross-sectional view of the bone anchor assembly.

FIG. 13 is a perspective view of a driving tool for inserting a bone anchor assembly into bone.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

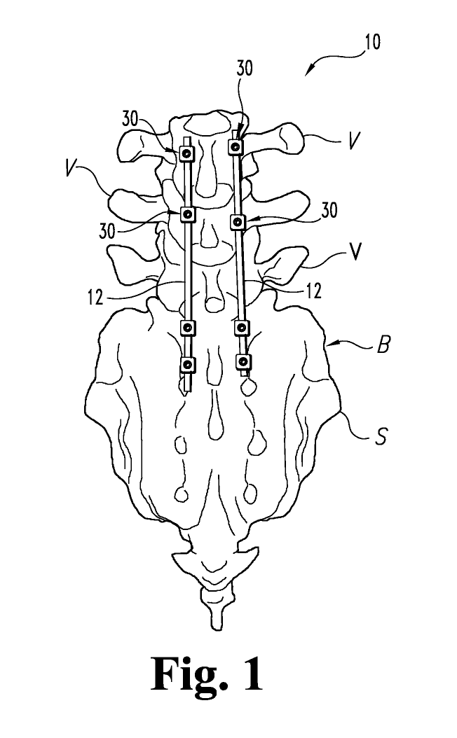

FIG. 1 illustrates a posterior spinal implant system 10 located along a spinal column of a patient. Implant system 10 generally includes several bone anchor assemblies 30 with at least one elongated connecting element 12 structured to selectively interconnect two or more bone anchors. Connecting elements 12 may be a spinal rod, plate, bar, or other elongated element having a length to extend between at least two vertebrae. Spinal implant system 10 may be used for, but is not limited to, treatment of degenerative spondylolisthesis, fracture, dislocation, scoliosis, kyphosis, spinal tumor, and/or a failed previous fusion. More specifically, in one embodiment implant system 10 is affixed to posterior elements, such as the pedicles of vertebra V, or other bones B of the spinal column segment, from a posterior approach. Bones B can include the sacrum S and/or one or more of several vertebrae V. Spinal implant system 10 can be engaged to vertebrae of one or more levels of the sacral, lumbar, thoracic and/or cervical regions of the spinal column. Other embodiments contemplate that the spinal implant system 10 is engaged along other portions of the spine, such as the anterior, lateral or oblique portions of the vertebrae V. Still other embodiments contemplate applications in procedures other the spinal stabilization procedures.

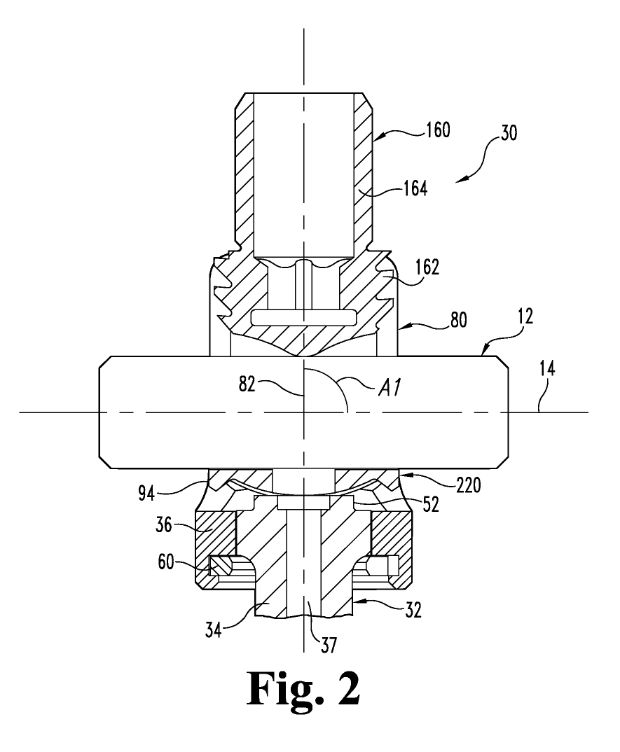

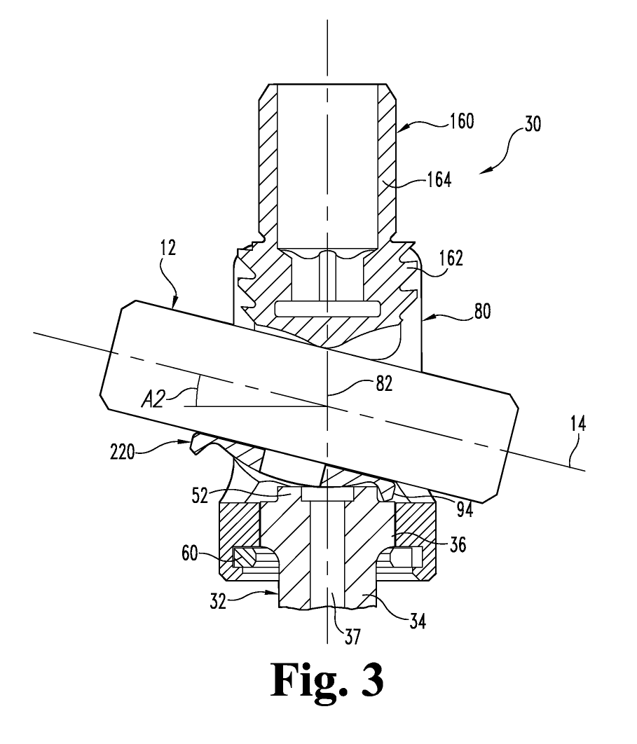

Referring to FIGS. 2-3, there is shown a longitudinal section view of a portion of one embodiment of bone anchor assembly 30 and connecting element 12 projecting from opposite sides of the bone anchor assembly 30. Bone anchor assembly 30 includes a bone anchor 32 with a distal bone engaging portion 34 configured for attachment to a vertebra, such as cervical, thoracic, lumbar and/or sacral vertebrae, or other bones or tissues in the body of a patient. Bone anchor assembly 30 also includes a receiver 80 coupled to bone anchor 32 with the retaining member 60. Receiver 80 includes a passage 94 extending through opposite sides of the receiver 80 that receives the connecting element 12 in a transverse orientation to the bone anchor 32.

An adjustable saddle 220 is located in the receiver 80 between a proximal head 36 of the bone anchor 32 and connecting element 12. Saddle 220 supports the connecting element 12 in the receiver 80 and pivots in a sagittal plane defined by the central longitudinal axis 14 of the connecting element 12 and a central longitudinal axis 82 of the receiver 80. Saddle 220 supports the connecting element 12 and maintains a proximal support surface of saddle 220 in contact with the connecting element 12 at various orientations of longitudinal axis 14 relative to the longitudinal axis 82 that vary from an orthogonal orientation A1, such as shown in FIG. 2, to a maximum angular orientation A2, such as shown in FIG. 3. In one embodiment, angle A2 is 30 degrees from the orthogonal orientation 14 of the connecting element 12. Other embodiments contemplate angle A2 ranging from more than 0 degrees to about 45 degrees. The orientation of the connecting element 12 and saddle 220 can vary at angle A2 relative to the longitudinal axis 82 while the orientation between the receiver 80 and bone anchor 32 is maintained in a fixed or substantially fixed relationship. As the saddle 220 is pivoted toward the maximum angular orientation, one end of the saddle member 220 can project outwardly from the passage 94 through the adjacent end of the receiver 80, as shown in FIG. 3. Engaging member 160 is engaged to the receiver 80 and secures the connecting element 12 in the receiver 80 against the saddle 220 in the selected or desired orientation. In one form, the saddle 220 is made from a commercially pure titanium (“CP—Ti”), but in other forms the saddle 220 can be manufactured from any suitable biocompatible material.

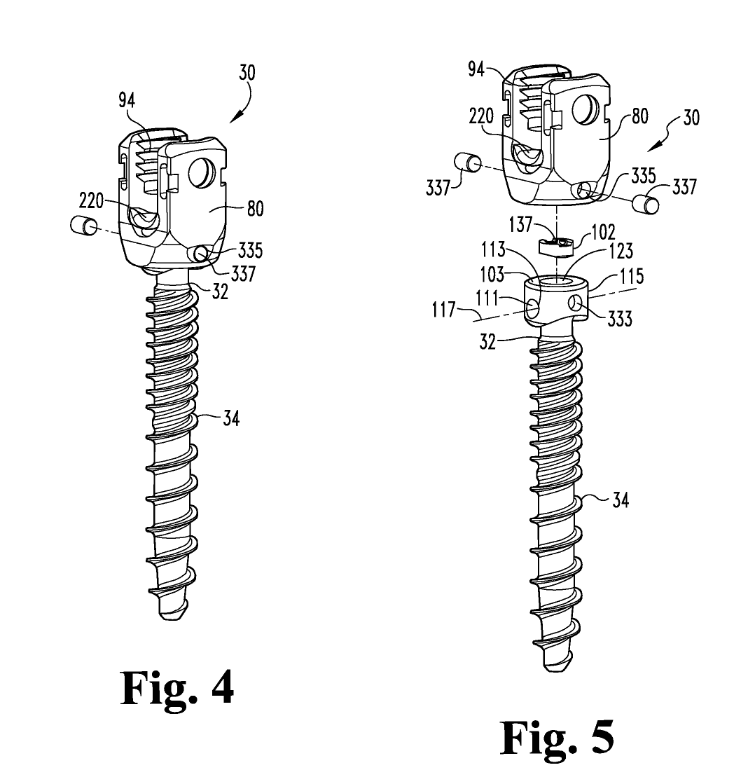

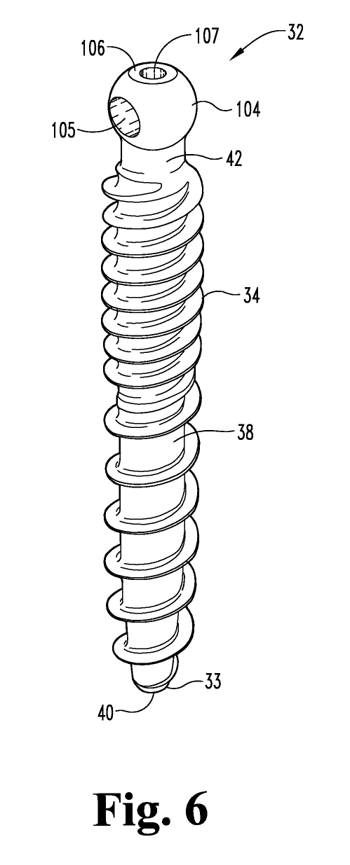

Referring collectively to FIGS. 4 and 5, wherein like numeral references correspond to similar elements, another representative bone anchor assembly 30 is illustrated without connecting element 12 or engaging member 160. As with the previous form, the bone anchor assembly 30 includes a receiver 80 that includes an adjustable saddle 220. Adjustable saddle 220 operates in the same manner as that described in connection with the form illustrated in FIGS. 2 and 3. As described in greater detail below, the bone anchor assembly 30 includes a spacer 102 that is received within a screw cap 103. As illustrated in FIG. 6, the bone anchor 32 includes a proximal screw head 104 that is generally circular or spherical in shape that extends upwardly along a vertical axis in relation to the bone engaging portion 34.

Bone anchor 32 also includes a passage 105 that runs horizontally through a central portion of the screw head 104. In addition, an upper portion 106 of the screw head 104 includes an aperture 107. In one form, bone anchor 32 is made from a titanium alloy, but in other forms the bone anchor 32 can be made from any other suitable biocompatible material. Referring collectively to FIGS. 5 and 6, the screw cap 103 pivotally connects to the screw head 104. In particular, a sub-assembly pin 111 is inserted through a first aperture 113 on a side surface 115 of the screw cap 103 and into and through the passage 105 running through the screw head 104 and then into a second corresponding aperture 113 on the opposite side surface 115 of the screw cap 103. Thus, the bone anchor 32 is capable of pivoting about a transverse or horizontal axis or plane 117 defined by the sub-assembly pin 111. In this form, the bone anchor assembly 30 is operable to pivot about transverse plane 117 and sagittal plane 14 thereby allowing for adjustment in two planes.

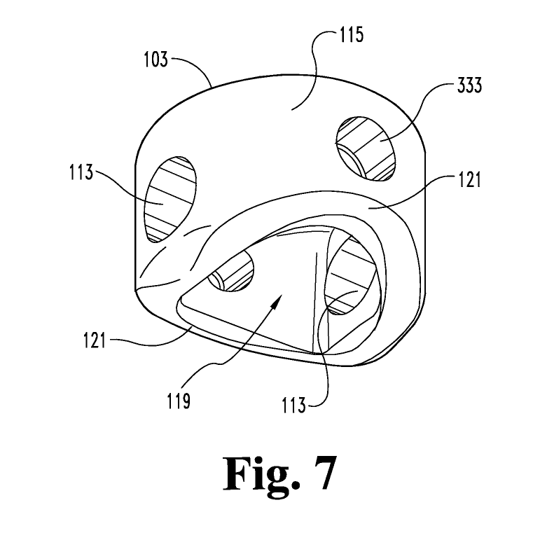

Referring collectively to FIGS. 5-8, as previously set forth, the screw cap 103 is sized and configured to pivotally be connected with the screw head 104 of the bone anchor 32. The side surface 115 of the screw cap 103 defines an interior cavity 119 that is sized and configured to receive the screw head 104. The screw head 104 is inserted into the interior cavity 119 such that apertures 113 in the side wall 115 are aligned with the passage 105 running through the screw head 104. Once properly aligned, the sub-assembly pin 111 is inserted through the apertures 113 and passage 105 thereby pivotally securing the screw cap 103 to the screw head 104. The side wall 115 of the screw cap 103 includes at least one cutout portion 121 thereby allowing the bone anchor 32 to pivot further on the screw head 104. In one form, the screw cap 103 and sub-assembly pin 111 are made from cobalt-chromium-molybdenum (“CCM”), but in other forms they can be manufactured from any biocompatible material.

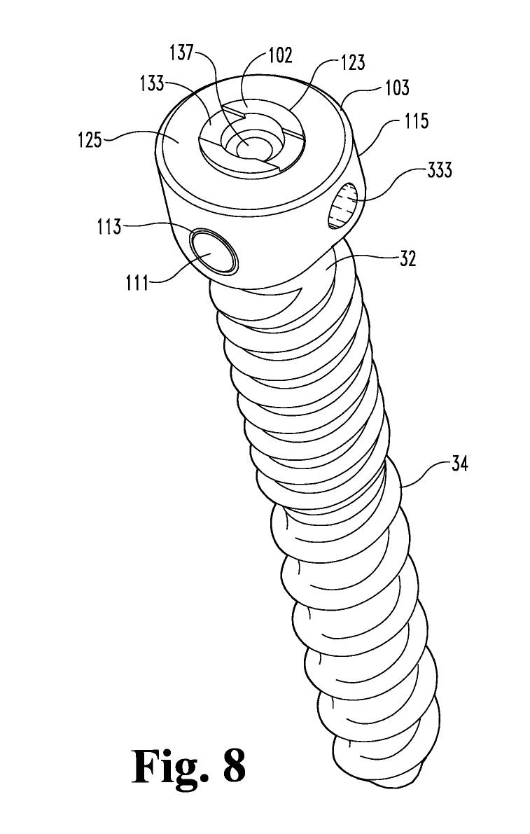

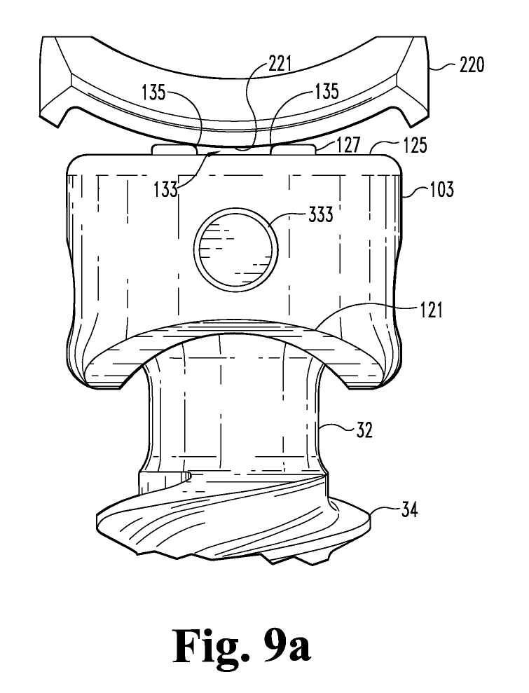

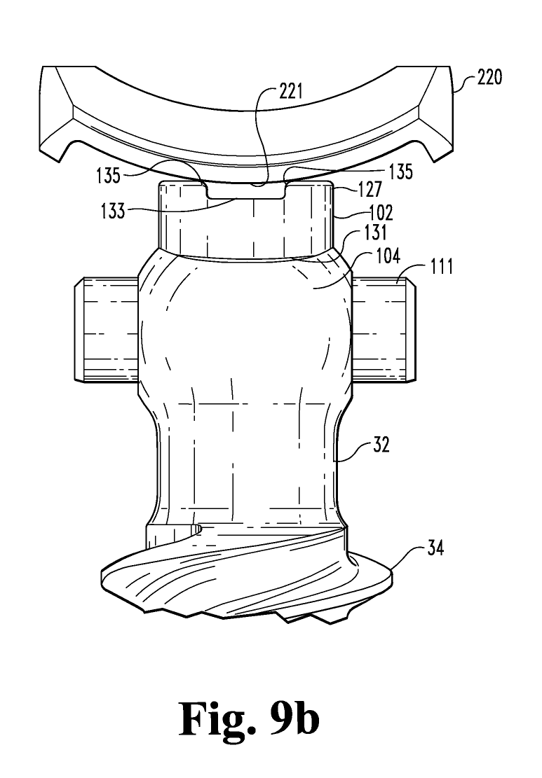

As illustrated in FIGS. 5 and 8, the screw cap 103 also includes an aperture 123 running through an upper surface 125 of the screw cap 103. In this form, the screw cap 103 has a generally cylindrical shape and the aperture 123 is generally circular in shape, however other shapes and configurations are contemplated in other forms. As previously set forth, the spacer 102 is sized and configured to be received within the aperture 123 in the upper surface 125 of the screw cap 103. As illustrated in FIGS. 9a and 9b, the spacer 102 is sized and configured such that an upper portion 127 of the spacer 102 protrudes outwardly and away from the upper surface 125 of the screw cap 103. The spacer 102 also includes a formed lower end 131 that is sized and configured to fit on the top of the screw head 104. In one form, the formed lower end 131 is in the form of a concave recess sized and configured to fit around an upper portion of the screw head 104.

The upper portion 127 of the spacer 102 includes a channel 133 that runs from opposing sides of the spacer 102. The channel 133 defines opposing edges 135 in the spacer 102. As illustrated in FIGS. 9a and 9b, the edges 135 are operable to bite into or fixedly engage a lower surface 221 of the saddle 220. In particular, the edges 135 are configured to bite into the saddle 220 when the engaging member 160 is tightened down onto connecting element 12 once positioned accordingly in the receiver 80 (see e.g. FIG. 2-3). In one form, the channel 133 is aligned in the spacer 102 to run substantially perpendicular to the sagital axis or plane 14. Referring to FIG. 5, a spacer passage 137 is included in a central portion of the spacer 102 to provide access to the aperture 107 in the screw head 104.

Referring to FIG. 6, the bone anchor 32 described herein can be included with bone engaging portion 34 configured as a bone screw, vertebral hook, bone clamp, and or other suitable bone engaging arrangement. Bone anchor 32 includes an elongated bone engaging portion 34 extending from a distal end portion 33 along a central longitudinal axis to the proximal head 104 that is centered on the central longitudinal axis. Bone engaging portion 34 is shown with an elongated shaft 38 having one or more threads along at least a portion thereof. The threads may be cancellous threads with the shaft sized and configured for implantation into a vertebra or other bone. The threads of the bone engaging portion 34 may be self-tapping, self-drilling, continuous, intermittent, of multiple thread forms, or other appropriate configurations. Furthermore, the bone anchor 32 may include a lumen 37 as shown in FIGS. 2-3, or be solid. Lumen 37 extends through the proximal and distal ends of the bone anchor 32 for receipt of guidewire and/or injection of material into the bone. One or more fenestrations may be provided along the bone engaging portion 34 of the bone anchor 32 that communicate with lumen 37.

Bone anchor 32 includes the elongated shaft 38 extending proximally and distally along the longitudinal axis between the head 104 and distal end portion 33. Distal end portion 33 includes a flat distal end 40, and tapers outwardly from the distal end 40 along a frusto-conically shaped end portion 33 to a threaded portion of the shaft 38. The threaded portion of the shaft 38 includes a helical thread with a major diameter and a minor diameter that are constant along a major length of the shaft 38 from the distal end portion 33 to a transition portion 42 between the shaft 38 and the screw head 104. Transition portion 42 includes a smooth and circular outer surface extending around shaft 38 that defines a third diameter that is greater than the minor diameter and less than major diameter. The helical thread runs out at transition portion 42. Other embodiments contemplate other configurations for the thread profile and shaft 38, including those with varying diameters and profiles along the length of shaft 38.

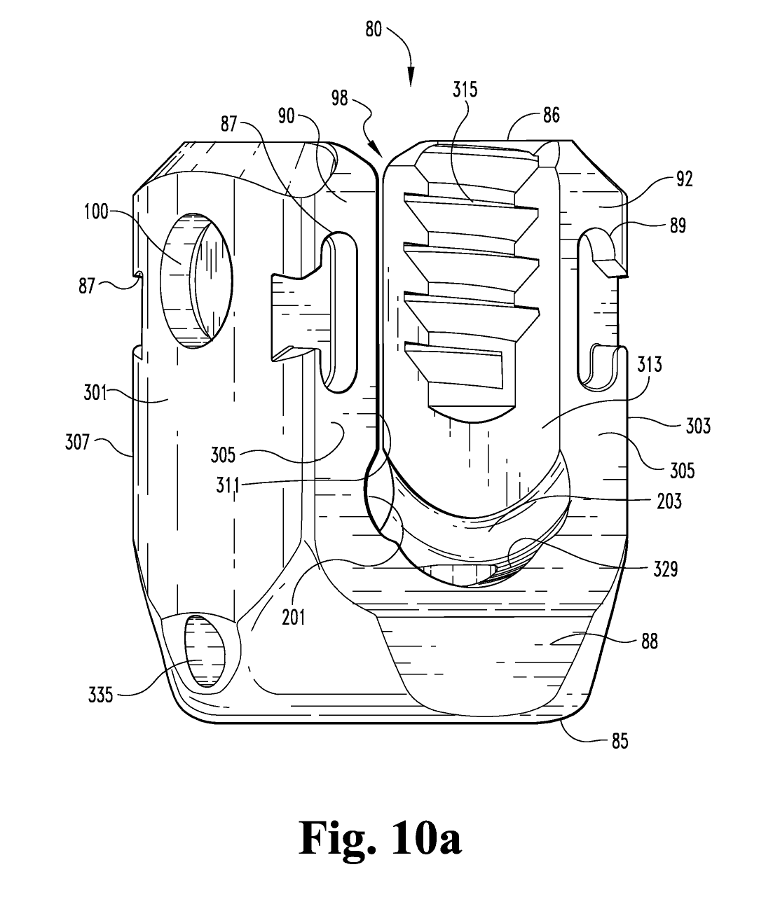

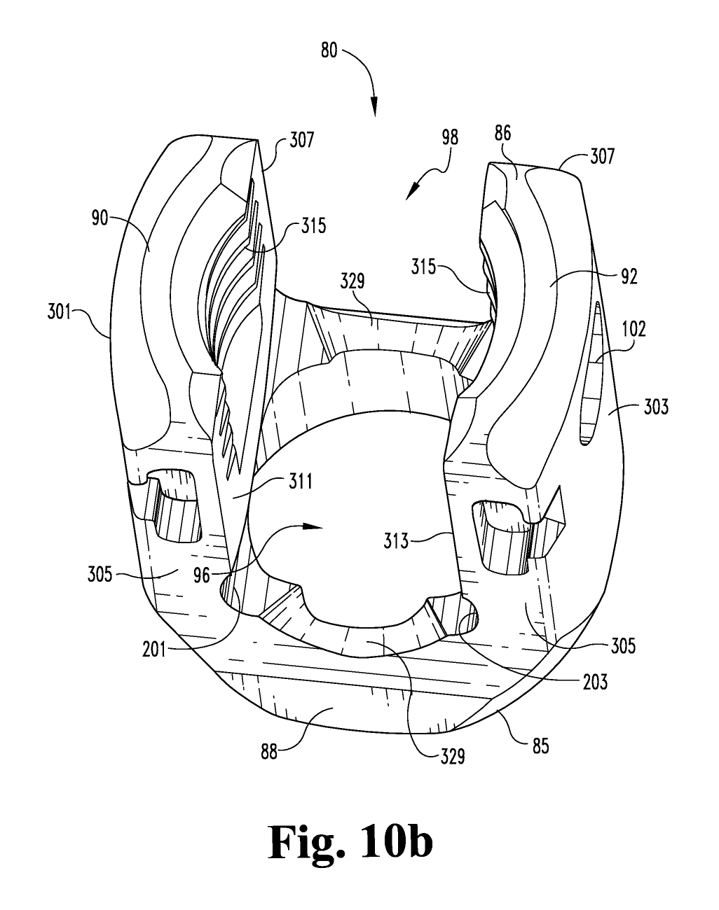

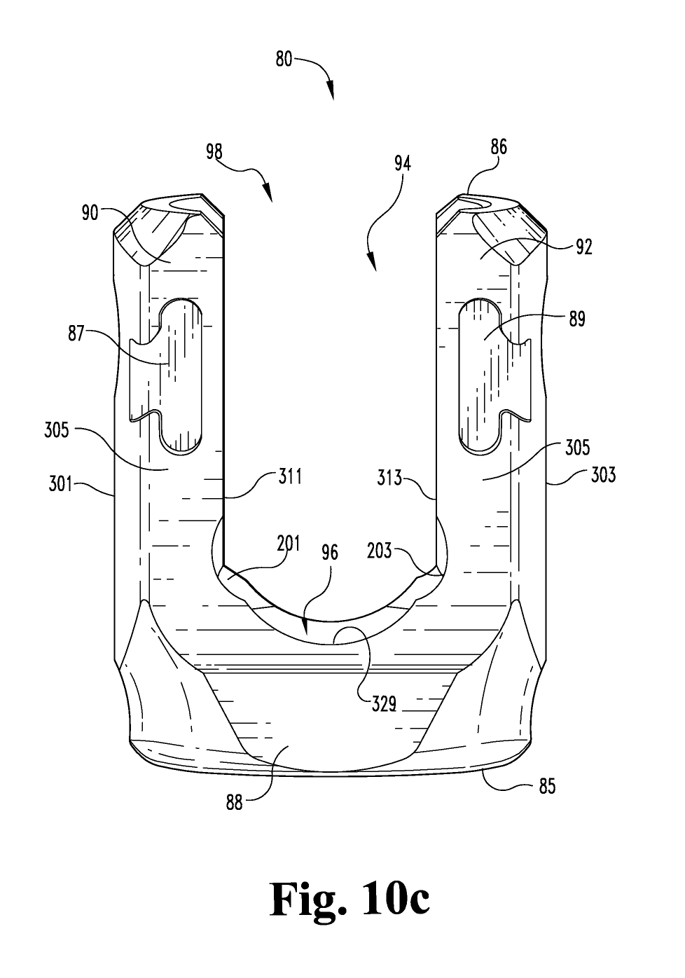

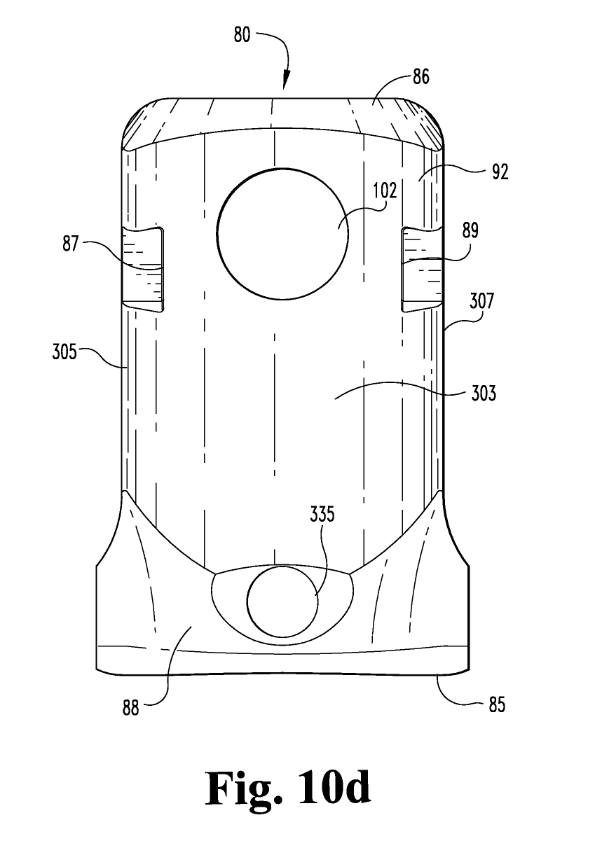

FIGS. 10a-10d show further details of the receiver 80. Receiver 80 includes a U-shaped body extending along a central longitudinal axis between a distal end 85 and a proximal end 86. Receiver 80 includes a distal portion 88 and a pair of arms 90, 92 extending proximally from the distal portion 88 on opposite sides of the longitudinal axis to the proximal end 86. Arms 90, 92 define a passage 94 therebetween that opens at opposite sides of arms 90, 92 to receive the connecting element 12 in a transverse orientation to the longitudinal axis of the receiver 80.

Distal portion 88 also includes a receptacle 96 that opens into passage 94 and extends from the passage 94 through the distal end 85. Arms 90, 92 also define a proximal opening 98 at proximal end 86 that extends the along arms 90, 92 to the passage 94. Engaging member 160 is engaged to the receiver 80 through the proximal end opening 98 to contact the connecting element 12 in passage 94. See e.g. FIGS. 2 and 3. Arms 90, 92 each include a circular recess 100, 102 in the outer side surface 301, 303, respectively, thereof that face opposite directions from one another. Arm 90 also includes oblong recesses 87 in each of the opposite end surfaces 305, 307 thereof. Arm 92 similarly includes oblong recesses 89 in each of the opposite end surfaces 305, 307 thereof. The recesses 87, 89 provide locations in which various tools and instrumentation can be engaged and mounted to the receiver 80 to facilitate implantation and maneuvering of the bone anchor 30 and connecting element 12 in the patient. End surfaces 305, 307 each are elongated in a longitudinal direction in a parallel orientation to the longitudinal axis of the receiver 80, and extend between the respective outer side surfaces 301, 303 to the respective inner surface 311, 313 of arms 90, 92 in an orthogonal orientation to the longitudinal axis of the receiver 80. Each of the inner surfaces 311, 313 includes a central concavely curved portion and linear end portions between the respective end surfaces 305, 307. The central concave portion of inner surface 311, 313 defines a thread profile 315 to threadingly engage the engaging member 160. Each thread profile 315 extends along the longitudinal axis of the receiver 80 from proximal end 86 of arms 90, 92 to a location adjacent to the passage 94 in the receiver 80.

Receiver 80 includes a bottom surface 329 that extends between the inner surfaces 311, 313 of arms 90, 92 along the distal side of the passage 94. Receptacle 96 opens through bottom surface 329. In addition, each of the arms 90, 92 includes a groove 201, 203, respectively, formed in the respective inner surface 311, 313 thereof along bottom surface 329. Each groove 201, 203 extends from one of the end surfaces 91 of the respective arm 90, 92 to the other end surface 93 of the respective arm 90, 92. As shown in FIG. 10a, each groove 201, 203 is curved between the respective end surfaces of the arm in which it is formed with the distal side of the groove defined by a radius so that the middle of the curved groove is located more distally than the opposite ends of the groove. The distal sides of the grooves 201, 203 intersect receptacle 96 adjacent the middle portions of the grooves 201, 203. Grooves 201, 203 extend from the middle portion thereof so that the opposite ends of grooves 201, 203 are spaced proximally from the bottom surface 329 where the groove exits at the opposite end surfaces 305 or end surfaces 307 of the respective arm 90, 92. Grooves 201, 203 are concavely curved in the respective inner surface 311, 313 to form a C-shape as shown in FIG. 10a. As previously set forth, saddle 220 is sized and configured to slide in grooves 201, 203 to allow for sagittal adjustment of the connecting element 12.

Referring to FIGS. 4, 5, and 10b, the receptacle 96 includes a generally square shaped opening through the bottom surface 329 extending around the proximal side of the receptacle 96. The screw cap 103 is sized and configured to be received in receptacle 96. The screw cap 103 includes a pair of opposing apertures 333 that are aligned with a pair of receiver apertures 335 in the receiver 80. As such, once the screw cap 103 is inserted into the receiver 80 through the receptacle 96, the apertures 333 of the screw cap 103 are positioned to be in alignment with the receiver apertures 335 in the receiver 80. At this point, a pair of connector pins 337 are inserted into the apertures 335 in the receiver 80 and then into the receiver apertures 333 thereby fixedly securing the receiver 80 to the bone anchor 32.

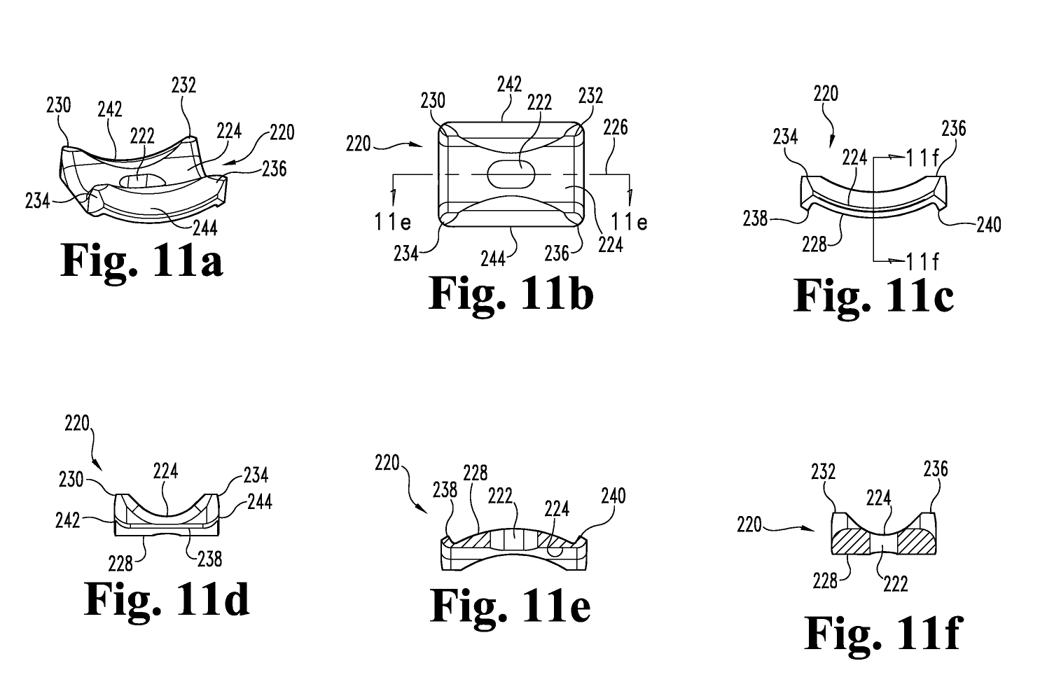

FIGS. 11a-11f show various view of saddle 220. Saddle 220, as shown in FIGS. 2-3 and 4-5, is positioned in receiver 80 between connecting element 12 and spacer 102. Saddle 220 includes a body with a rectangular shape when saddle 220 is viewed in a proximal to distal direction, as shown in FIG. 11b. Saddle 220 extends along a longitudinal axis 226 between opposite ends, and includes an oblong hole 222 extending through a center thereof between upper and lower surfaces thereof. Hole 222 aligns with lumen 37 of bone anchor 32. The oblong shape of hole 222 allows at least a portion of hole 222 to align with lumen 37 even if saddle 220 is pivoted to a non-centered position in receiver 80. Saddle 220 includes a proximal support surface 224 against which connecting element 12 is positioned. Proximal support surface 224 is linear in a direction paralleling longitudinal axis 226 as shown in FIG. 11b and is concavely curved orthogonally to longitudinal axis 226 as shown in FIG. 11f. The shape of proximal support surface 224 matches the shape of the portion of the outer surface of connecting element 12 positioned thereagainst.

Saddle 220 includes a distal surface 228 opposite proximal support surface 224. Distal surface 228 is convexly curved along longitudinal axis 226 as shown in FIG. 11c, and is linear between the opposite sides of saddle 220. Distal surface 228 contacts and is supported by the spacer 102. The convexly curved distal surface 228 facilitates pivoting movement of saddle 220 in the plane that includes the longitudinal axis of the receiver 80 and the longitudinal axis 14 of connecting element 12. In addition, saddle 220 includes ears 230, 232, 234, 236 extending outwardly from proximal support surface 224 that are received in respective ones of the grooves 201, 203 of receiver 80. Ears 230, 232 are positioned in and translate along groove 118, and ears 234, 236 are positioned in and translate along groove 120 as saddle 220 pivots in receiver 80 in the plane defined by longitudinal axis 82 of receiver 80 and longitudinal axis 14 of connecting element 12. Ears 230, 232, 234, 236 maintain saddle 220 within receiver 80 along a path defined by the grooves 201, 203 and prevent saddle 220 from pivoting or twisting to an undesired orientation in the receiver 80.

Saddle 220 also includes a first tooth 238 at one end thereof that extends between ears 230, 234 and projects distally from distal surface 228, and saddle 220 includes a second tooth 240 at the opposite end thereof that extends between ears 232, 236 and projects distally from distal surface 228. Saddle 220 includes a first elongate side 242 extending between ears 230, 232 with a proximal side that is concavely curved between ears 230, 232 and an opposite convexly curved distal side between ears 230, 232. Saddle 220 also includes a second elongate side 244 extending between ears 234, 236 with a proximal side that is concavely curved between ears 234, 236 and an opposite convexly curved distal side between ears 234, 236. The curvature of sides 242, 244 corresponds to the curvature of the respective groove 201, 203 so that saddle 220 extends across bottom surface 116 of receiver 80 into the grooves 201, 203. Convexly curved distal surface 228 contacts rails 54 of platform 52 and slides along rails 54 as saddle 220 translates in grooves 201, 203. When saddle 220 is sufficiently pivoted in receiver 80 to a maximum angle, one of the first and second teeth 238, 240 contacts an adjacent side of the platform 52 to prevent further pivoting movement of saddle 220 in receiver 80, as shown in FIG. 3.

Referring to FIGS. 2-3, the engaging member 160 is movably engaged to the arms 90, 92 of the receiver 80 through the proximal end opening 98 of the receiver 80. Engaging member 160 is movable toward the passage 94 by threading it along arms 90, 92 of the receiver 80 to contact the connecting element 12 and direct the connecting element 12 into the receiver 80 and into engagement with the proximal support surface 224 of the saddle 220, which in turn moves and/or forces the distal surface 228 of the saddle 220 into contact with the platform 56 of the bone anchor 32, securing the connecting element 12 and bone anchor 32 to one another and securing the bone anchor 32 against the retaining member 60 of the receiver 80. In the illustrated embodiment, the engaging member 160 is a set screw type element with an externally threaded body 162 that threadingly engages the inner threads 315 provided along the arms 90, 92. Other embodiments contemplate an engaging member in the form of a nut, cap, or combination of nut and setscrew. In still other embodiments, the engaging member 160 engages the receiver 80 in a non-threaded manner, such as a friction fit, interference fit, or bayonet lock. Engaging member 160 also includes a proximal break-off portion 164 extending from the body 162 to facilitate engagement of the engaging member 160 to the receiver 80 and in the application of sufficient force to secure the assembly of the connecting element 12 against the saddle 220 and the saddle 220 against the bone anchor 32. Break-off portion 164 is severed upon application of a threshold torque that provides the desired level of fixation of the anchor assembly 30.

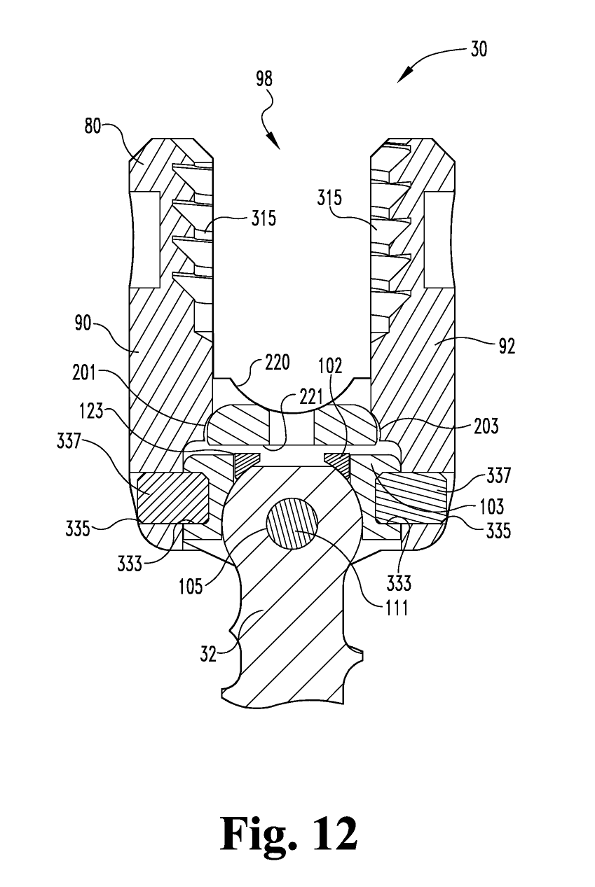

Referring to FIG. 12, a cross-sectional view of the bone anchor assembly 30 is illustrated. As illustrated, the sub-assembly pin 111 has been inserted through the apertures 113 of the screw cap 103 and into the passage 105 of the bone anchor 32 thereby securing the screw cap 103 to the bone anchor 32. The spacer 102 has been inserted into the aperture 123 of the screw cap 103 and is positioned on an upper surface of the screw head 104. Pins 337 have been inserted through the passages 335 in the receiver 80 and into the apertures or passages 333 of the screw cap 103 thereby securing the bone anchor 32 to the receiver 80. As previously set forth, the bone anchor 32 is allowed to pivot in the screw cap 103 along a transverse or horizontal axis.

As discussed earlier, the spacer 102 is sized and configured to engage a lower surface 221 of the saddle 220 thereby securing the saddle 220 in a locked orientation once the connecting member 12 and engaging member 160 have been positioned in the receiver 80. The force applied by the engaging member 160 to the connecting member 12 when it is tightened in the receiver 80 causes the saddle 220 to engage the spacer 102 thereby locking the saddle 220 in a respective position. As previously set forth, the spacer 102 includes edges 135 that are configured to bite into the lower surface 221 of the saddle 220.

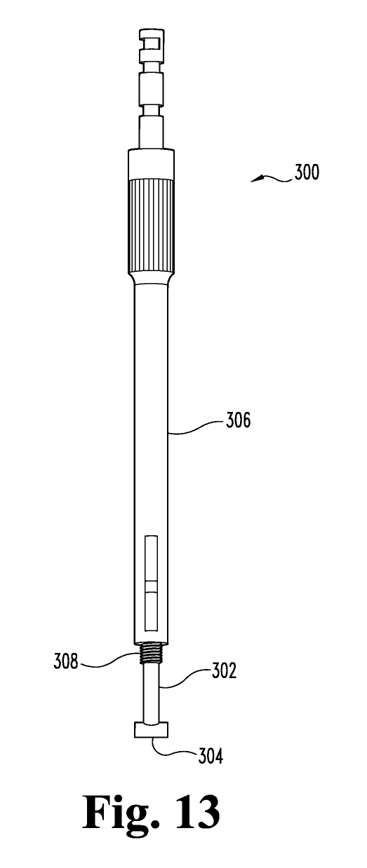

FIG. 13 shows a driver instrument 300 that can be used to insert and drive the anchor assemblies herein into bone. Driver instrument 300 includes an inner shaft 302 with a distal, cylindrically shaped foot 304 that is elongated along an axis transverse to the longitudinal axis of the instrument to fit within the passage 94 of the anchor 30 against the saddle 220. Driver instrument 300 also includes an outer tubular member 306 positioned around the inner shaft 302. Tubular member 306 includes a distal collar 308 spaced proximally from the foot 304. Tubular member 306 is rotatable relative to inner shaft 302 to threadingly engage the collar 308 with the arms 90, 92 of the receiver 80 with the foot 304 in the receiver 80, and press the foot 304 against the saddle 220 to secure it against the head 104 of the bone anchor 32. The anchor assembly 30 can then be rotated and threaded into the bone as a rigid, assembled construct with the driver instrument 300.

As previously set forth, as engaging member 160 is threaded down on connecting element 12, connecting element 12 exerts a force on the saddle 220. The saddle 220 in turn exerts a force on the upper surface of the spacer 102. The lower surface of spacer 102 will then exert a force on the head 104. Thus, engaging the member 160 locks the connecting element 12 in place within the saddle 220 of the receiver 80 along a predetermined sagittal plane and the spacer 102 locks the head 104 in a predetermined transverse plane. In addition, edges 135 of the spacer 102 can bite into the saddle 220 to further help secure the saddle 200 in its relative position.

The bone anchor assemblies discussed herein allow adjustment of the angle of the saddle and thus the angle of the connecting element extending through the saddle in a single plane defined by the longitudinal axis of the connecting element and the longitudinal axis of the receiver. The bone anchor assemblies provide a two-piece construct for the receiver and the bone anchor that forms a rigid or semi-rigid bone anchor assembly when the receiver is assembled with the bone anchor while limiting angulation of the saddle in a particular plane. The two piece construct allows the receiver and bone anchor to be comprised of different materials suitable for the expected loading of the components. For example, the receiver can be made from a higher strength material than the material for the bone anchor so that the splaying and other deformations of the receiver can be limited by the higher strength material and so that the side of the receiver can be minimized to limit intrusiveness into the surrounding tissue post-implantation.

Materials for the anchors, receivers, saddles, spacers and engaging members disclosed herein can be chosen from any suitable biocompatible material, such as titanium, titanium alloys, cobalt-chromium, cobalt-chromium alloys, or other suitable metal or non-metal material. Connecting element 12 can be made from the same material as one or more of the components of the anchor assembly to which it is engaged, or from a different material. For example, connecting element 12 can be made from PEEK, plastic, titanium or titanium alloy, cobalt-chrome, composite material, or other material that is the same or different from the material of one or more components of the anchor assembly to which is engaged. The anchor assemblies can be sized for placement at any level of the spine and for engagement with any bony portion of the spine. In one particular embodiment, the anchor assemblies are engaged to pedicles of the vertebrae. Of course, it is understood that the relative size of the components of the anchor assemblies can be modified for the particular vertebra(e) to be instrumented and for the particular location or structure of the vertebrae to which the anchor assembly will be engaged.

Although various embodiments have been described as having particular features and/or combinations of components, other embodiments are possible having a combination of any features and/or components from any of embodiments as discussed above. As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the terms “proximal” and “distal” refer to the direction closer to and away from, respectively, an operator (e.g., surgeon, physician, nurse, technician, etc.) who would insert the medical implant and/or instruments into the patient. For example, the portion of a medical instrument first inserted inside the patient's body would be the distal portion, while the opposite portion of the medical device (e.g., the portion of the medical device closest to the operator) would be the proximal portion.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. A bone anchor assembly, comprising:
a receiver extending along a central longitudinal axis between a proximal end and an opposite distal end, said receiver including a distal portion defining a receptacle opening at said distal end and a pair of arms extending from said distal portion along said central longitudinal axis;
a bone anchor including a distal bone engaging portion and a head at a proximal end of said distal bone engaging portion;
a cap including an upper surface extending to a lower surface, said lower surface including an interior cavity sized and configured to receive said head of said bone anchor such that said bone anchor can pivot along a first axis, said cap being positioned in said receptacle of said receiver; and
a spacer positioned in an aperture in said cap such that an upper portion of said spacer protrudes outwardly from said cap and a lower portion of said spacer is connected with said head.

2. The bone anchor assembly of claim 1, further comprising a saddle positioned in said passage of said receiver adjacent to a bottom surface of said receiver, said saddle including a proximal support surface and a distal surface opposite said proximal support surface.

3. The bone anchor assembly of claim 2, wherein said upper portion of said spacer is in contact with said distal surface of said saddle.

4. The bone anchor assembly of claim 3, wherein said spacer includes at least one edge operable to bite into said distal surface of said saddle to secure said saddle in a respective location within said receiver.

5. The bone anchor assembly of claim 2, wherein said saddle is movable in said receiver so that said support surface parallels a longitudinal axis of a connecting element in orientations of said longitudinal axis of said connecting element that vary up to 30 degrees from an orthogonal orientation of said longitudinal axis of said connecting element with said central longitudinal axis of said receiver.

6. The bone anchor assembly of claim 1, wherein said cap includes a first set of apertures sized and configured to receive a first pin, wherein said head of said bone anchor includes a passage aligned with said first set of apertures and said first pin is inserted through said first set of apertures and said passage thereby securing said cap to said bone anchor.

7. The bone anchor assembly of claim 6, wherein said bone anchor pivots along said first axis by pivoting about said first pin.

8. The bone anchor assembly of claim 6, wherein said cap includes a second set of apertures and said distal portion of said receiver includes a third set of apertures, wherein a second pin is inserted through a respective one of said second set of apertures and a respective one of said third set of apertures and a third pin is inserted through a respective one of said second set of apertures and said third set of apertures thereby securing said cap to said receiver.

9. A bone anchor assembly, comprising:
a receiver extending along a central longitudinal axis between a proximal end and an opposite distal end, said receiver including a distal portion defining a receptacle opening at said distal end and a pair of arms extending from said distal portion along said central longitudinal axis on opposite sides of a passage, said receiver including a bottom surface extending along said passage between said pair of arms, wherein said passage opens at opposite sides of said receiver between said pair of arms and said receptacle opens into said passage through said bottom surface;
a cap having a cavity located in a central portion of said cap;
a bone anchor including a distal bone engaging portion and a head at a proximal end of said distal bone engaging portion, said head being configured to be received in said cap through said cavity;
a saddle positioned in said passage of said receiver adjacent to said bottom surface of said receiver, said saddle including a proximal support surface and a distal surface opposite said proximal support surface;
a spacer positioned between said saddle and said head of said bone anchor;
a connecting element extending along a central longitudinal axis, said connecting element being located in said passage and extending through said opposite sides of said receiver; and
an engaging member engaged to said pair of arms to secure said connecting element against said proximal support surface of said saddle, wherein said saddle engages said receiver and is limited to movement in said receiver in a single plane defined by said central longitudinal axis of said receiver and said central longitudinal axis of said connecting element while said bone engaging portion remains in said first orientation.

10. The bone anchor assembly of claim 9, wherein said saddle is movable in said receiver so that said support surface parallels said longitudinal axis of said connecting element in orientations of said longitudinal axis of said connecting element that vary up to 30 degrees from an orthogonal orientation of said longitudinal axis of said connecting element with said central longitudinal axis of said receiver.

11. The bone anchor assembly of claim 9, wherein:
said pair of arms include inner surfaces facing one another on opposite sides of said passage;
said inner surfaces each include a groove formed therein that is curved between opposite ends of a respective one of said pair of arms so that said curve includes a most distal portion at said central longitudinal axis and said groove is curved proximally from said most distal portion toward said opposite ends of said respective arm; and
said saddle including at least one ear on each side of said proximal support surface that are positioned in a respective one of said grooves, said ears being slidably movable along said respective one of said grooves.

12. The bone anchor assembly of claim 11, wherein said saddle includes a pair of ears extending from each side thereof with each of said ears of said side located at an end of said saddle and each of said sides is concavely curved between said pair of ears thereof.

13. The bone anchor assembly of claim 9, wherein said cap includes a first set of apertures and said head of said bone anchor includes a passage, wherein a first pin is inserted through said first set of apertures and said passage to pivotally secure said bone anchor to said cap.

14. The bone anchor assembly of claim 13, wherein said cap includes a second set of apertures and said distal portion of said receiver includes a pair of opposing apertures, wherein when said cap is placed in said receptacle a second pin is placed in one of said pair of opposing apertures and a respective one of said second set of apertures and a third pin is placed in the other one of said pair of opposing apertures and the other respective one of said second set of apertures thereby securing said cap in said receptacle.

15. The bone anchor assembly of claim 9, wherein as said engaging member is tightened said saddle forcibly engages said spacer thereby causing said spacer to fixedly secure said head of said bone anchor in a transverse position.

16. The bone anchor assembly of claim 15, wherein said spacer includes at least one edge that is configured to bite into a lower surface of said saddle.

17. A bone anchor assembly, comprising:
a receiver extending along a central longitudinal axis between a proximal end and an opposite distal end, said receiver including a distal portion defining a receptacle opening at said distal end and a pair of arms extending from said distal portion along said central longitudinal axis on opposite sides of a passage with said passage opening at opposite sides of said receiver, said receiver including a bottom surface extending along said passage between said pair of arms, wherein said receptacle opens into said passage;
a cap having an upper surface extending down toward a lower surface, said cap having an interior cavity in said lower surface;
a bone anchor including a distal bone engaging portion and a head at a proximal end of said distal bone engaging portion, said head being positioned in said cavity of said cap such that said bone anchor can pivot along a transverse plane;
a saddle positioned in said passage of said receiver adjacent to said bottom surface of said receiver, said saddle including a proximal support surface and a distal surface opposite said proximal support surface;
a spacer positioned in said receptacle of said receiver such that an upper surface of said spacer is in contact with a lower surface of said saddle and a lower surface of said spacer is in contact with an upper surface of said head;
a connecting element extending along a central longitudinal axis, said connecting element being located in said passage and extending through said opposite sides of said receiver; and
an engaging member engaged to said pair of arms to secure said connecting element against said proximal support surface of said saddle, wherein force applied to said saddle is transferred to said spacer which in turn transfers force to said head of said bone anchor thereby preventing said bone anchor from further pivoting in said cap.

18. The bone anchor assembly of claim 17, wherein said cap includes a set of opposing apertures and said head of said bone anchor includes a passage, wherein a first pin is inserted through said first set of opposing apertures and said passage thereby pivotally securing said bone anchor to said cap.

19. The bone anchor assembly of claim 18, wherein said cap includes a second set of apertures and said distal portion of said receiver includes a pair of opposing apertures, wherein when said cap is placed in said receptacle a second pin is placed in one of said pair of opposing apertures and a respective one of said second set of apertures and a third pin is placed in the other one of said pair of opposing apertures and the other respective one of said second set of apertures thereby securing said cap in said receptacle.

20. The bone anchor assembly of claim 17, wherein said saddle is movable in said receiver so that said support surface parallels said longitudinal axis of said connecting element in orientations of said longitudinal axis of said connecting element that vary up to 30 degrees from an orthogonal orientation of said longitudinal axis of said connecting element with said central longitudinal axis of said receiver.
